(19) United States
(12) Patent Application Publication
(10) Pub. No.: US 2007/0287674 A1
(43) Pub. Date: Dec. 13, 2007

(54) New treatment of diabetes mellitus

(75) Inventors: Viqar Uddin Ahmad, Karachi (PK); Nasir Rasool, Sahiwal (PK); Muhammad Iqbal Choudhary, Karachi (PK); Shamsun Nihar Khan, Dhaka (BD)

Correspondence Address: SARFARAZ K. NIAZI, 20 RIVERSIDE DRIVE, DEERFIELD, IL, 60015, US

(73) Assignee: HEJ RESEARCH INSTITUTE OF CHEMISTRY, Karachi (PK)

(21) Appl. No.: 11/309,013

(22) Filed: Jun. 8, 2006

(51) Int. Cl. A61K 31/7028; A61P 3/10

(52) U.S. Cl. 514/26

(57) ABSTRACT

This invention is directed to a novel method for the treatment of diabetes mellitus (Type I, Impaired Glucose Tolerance [“IGT”] and Type II) by administering a therapeutically effective amount of one or both α-glucosidase inhibitor namely paniculoside IV and ent-16, 17-dihydroxy-(−)-kauran-19-oic acid to humans and animals.

### BACKGROUND

Glucosidase enzymes are involved in several biological processes such as the intestinal digestion, the biosynthesis of glycoproteins and the lysosomal catabolism of the glycoconjugates (Homonojirimycin isomers and N-alkylated homonojirimycins: structural and conformational basis of inhibition of glycosidases. Asano N, Nishida M, Kato A, Kizu H, Matsui K, Shimada Y, Itoh T, Baba M, Watson A A, Nash R J, Lilley P M, Watkin D J, Fleet G W., J Med Chem, 1998 Jul. 2; 41(14):2565-71). Intestinal α-glucosidases are involved in the final step of the carbohydrate digestion to convert these into monosaccharides which are absorbed from the intestine. Details of how starch is converted to glucose and absorbed is shown in Scheme 1.

### Scheme 1

Scheme 1. Schematic diagram of enzymatic degradation of poly and oligosaccharides and sucrose by Intestinal α-glucosidase.

As a result of the catalysis produced by α-glucosidase enzyme in the final step in the digestive process of carbohydrates, its inhibitors can retard the uptake of dietary carbohydrates and suppress postprandial hyperglycemia, and could be useful to treat diabetic and/or obese patients [Novel α-glucosidase Inhibitors with a tetrachlorophthalimide Skeleton., S. Sou, S. Mayumi, H. Takahashi, R. Yamasak, S. Kadoya, M. Sodeoka, and Y. Hashimoto, Bioorg. Med. Chem. Lett., 2000, 10, 1081].

The α-glucosidase inhibitors are effective in lowering the insulin release, insulin requirement and some can lower plasma lipids. The acarbose is a very widely prescribed drug in the management of the type II diabetes and recently a U.S. Pat. No. 6,387,361 to Rosner describes the use of acarbose in the treatment of obesity. According to the criteria issued by WHO (World Health Organization) based on a glucose tolerance test, diabetes mellitus and impaired glucose tolerance (hereinafter sometimes referred to as IGT) are distinguished by the fasting blood glucose level and the blood glucose level 2 hours after glucose loading. Patients with IGT have high blood glucose levels compared to those of patients with diabetes mellitus, and are reported to be at increased risk of developing diabetes mellitus and complications of arteriosclerotic diseases. In particular, it is known that patients with IGT who have blood glucose levels of 170 mg/dl or above at 2 hours following glucose loading, i.e., patients with high-risk IGT, may develop diabetes mellitus at a high rate [Diabetes Frontier, p. 136, 1992]. With regard to voglibose which is an α-glucosidase inhibitor, there are reports of studies on effects of voglibose for insulin-resistant IGT and diabetes [Yakuri-to-Chiryo (Japanese Pharmacology & Therapeutics), 24 (5):213 (1996); Metabol. Exp. Clin., 45:731, 1996]. Voglibose (AO-128) is also known to have effects of lowering blood glucose level and improving glucose tolerance in rats [Yakuri-to-Chiryo (Japanese Pharmacology & Therapeutics), 19 (11):161 (1991); Journal of Nutrition Science and Vitaminology, 45 (1): 33 (1992)]. On the contrary, it has also been reported that the effect of voglibose in improving glucose tolerance could not be verified in human [Rinsho-Seijinbyo, 22 (4): 109 (1992)]. An antibiotic pradimicin Q as α-glucosidase inhibitor is described in the U.S. Pat. No. 5,091,418 to Swada.

In addition, they have also been used as antiobesity drugs, fungistatic compounds, insect antifeedents, antivirals and immune modulators [Glycosidase inhibitors and their chemotherapeutic value, Part 1. el Ashry E S, Rashed N, Shobier A H., Pharmazie. 2000 April; 55(4):251-620]. The antiviral activity due to inhibition of α-glucosidase results form abnormal functionality of glycoproteins because of incomplete modification of glycans. Suppression of this process is the basis of antiviral activity [A glucosidase-Inhibitors as potential broad based antiviral agents, Anand Mehta, Nicole Zitzmann, Pauline M. Rudd, Timothy M. Block, Raymond A. Dwek, Febs Letters 430 (1998)17-22] and decrease in growth rate of tumors [Inhibition of experimental metastasis by an alpha-glucosidase inhibitor, 1,6-epi-cyclophellitol. Atsumi S, Nosaka C, Ochi Y, Iinuma H, Umezawa K. Cancer Res. 1993 Oct. 15; 53(20):4896-9]. The α-glucosidase inhibitor N-(1,3-dihydroxy-2-propyl)valiolamine is described as a promoter of calcium absorption in the U.S. Pat. No. 5,036,081.

In the present invention is reported a surprising discovery was made when it was discovered that the diterpenes paniculoside IV (16, β-17-hydroxy-ent-1-α-auzan 19-O-D-glucopyranosyl ester) and ent-16,17-acetonyl-(−)-kauran-19-oic acid obtained by the acid and basic hydrolysis of pulicarside 1, which was obtained from a plant source, Pulicaria undulata (herb) that belongs to the family Asteraceae (Compositae). This activity of the two listed dieterpenes as inhibitors of glucosidase enzyme has never before reported in the prior art.

### DETAILED DESCRIPTION

Pulicaria undulata (herb) belongs to the family Asteraceae (Compositae), which is a largest family of flowering plants. Plants of this family are found in frigid, temperate subtropical and tropical zones of Africa and Asia. The genus Pulicaria has eleven species, distributed in tropical and temperate regions of Pakistan [Flora of West Pakistan, E. Nasir, 1972, no. 20, pp. 770]. The plants of this genus are used in traditional medicine as tonic and substituted for tea, antispasmodic, hypoglycemic and as ingredients of perfume [D-Carvotanacetone from Pulicaria Undulata, Kamal E I Din A, Yousif G, Ishag K E, E I Egami A A, Mahmoud E N, Abu A I Futuh I M. Fitoterapia. 1992; 63:281] Aerial parts of Pulicaria undulata are used for antibacterial purpose [Antibacterial Properties of Essential Oils from Nigella Sativa Seeds (Cymbopogon Citratus) Leaves and Pulicaria Undulata Aerial Parts., Kamali H H, Ahmed A H, Mohammed A S, Yahia A A M, E I Tayeb I, Ali A A, Fitoterapia, 1998; 69:77-78]. Literature survey showed some reports on essential oils [isolation and antimicrobial activity of two phenolic compounds from Pulicaria odora L. Ezoubeiri A, Gadhi C A, Fdil N, Benharref A, Jana M, Vanhaelen M., J Ethnopharmacol. 2005 Jun. 3; 99(2):287-92.], terpenoids and flavonoids [Isolation of dihydroflavonol from Pulicaria undulata (L.) Kostel. Khafagy S M, Metwally A M, Omar A A., Pharmazie 1976; 31(9):649]

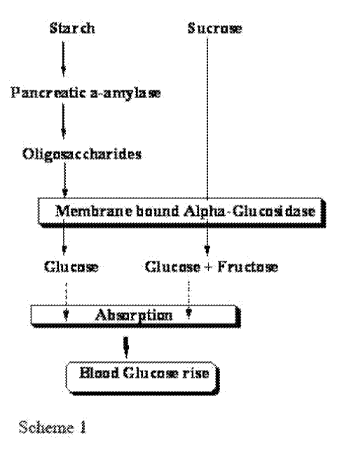

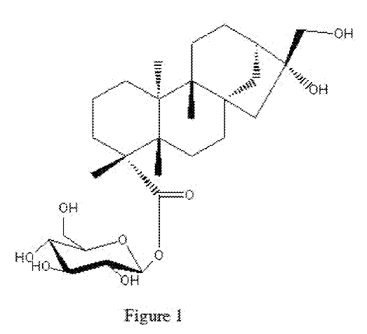

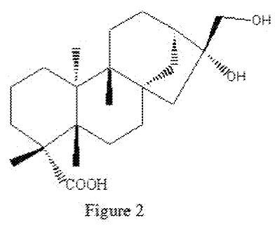

The present invention deals with the characterization of a paniculoside IV (1) and ent-16,17-dihydroxy-(−)-kauran-19-oic acid (2) [X. Jiang, M. Yunbao, X. YunIong, Phytochemistry 1992, 31, 917]. Chemical structures of these two compounds are shown in FIGS. 1 and 2.

### FIG. 1

FIG. 1 Structure of compound 1 [paniculoside IV]

### FIG. 2

FIG. 2. Structure of compound 2[ent-16,17-dihydroxy-(−)-kauran-19-oic acid]

### EXPERIMENTAL

General Analytical Instrumentation: TLC: Kieselgel F₂₅₄ (0.25 mm: Merck). Column chromatography (CC): silica gel (70-230 mesh; Merck), flash chromatography (FC): silica gel (230-400 mesh; Merck). Optical rotation: Jasco DIP-360 digital polarimeter. UV Spectra: Hitachi-UV-3200 spectrophotometer. IR spectra: Jasco-320-A spectrophotometer. ¹H-NMR, ¹³C-NMR, COSY, HMQC and HMBC Spectra: Bruker spectrometer. EI-MS and FAB-MS spectra: JMS-HX-110 spectrometer.

The shade-dried ground plant material (whole plant) of Pulicaria undulata L. (Asteraceae) was exhaustively extracted with methanol at room temperature. The extract was evaporated and dissolved in water and partitioned with hexane, chloroform, ethyl acetate and n-butanol. The ethyl acetate soluble extract was subjected to column chromatography (silica gel, Hexane/CHCl₃ mixtures of increasing polarity, CHCl₃, CHCl₃/MeOH mixtures of increasing polarity) and fifteen fractions (1-15) were collected. Pulicarside 1 was obtained from Fr. 8 when it was subjected to FC (silica gel, CHCl₃/MeOH (10:90)). Compound 1 was obtained from Fr. 9 when it was subjected to FC (silica gel, CHCl₃/MeOH (15:85). Compound 1 was also obtained when pulicarside 1 as subjected to acid hydrolysis: pulicarside 1 was refluxed with 0.5 N HCl for 2 h. After neutralization with NH₄OH, it was extracted with n-butanol. The n-butanol fraction was evaporated under reduced pressure to give glucoside without acetonyl moiety, ¹H-NMR data of which were identical with paniculoside IV (compound 1) (16, β-17-hydroxy-ent-1-α-auzan 19-O-D-glucopyranosyl ester [K. Yamasaki, H. Kohada, T. Kobayashi, N. Kaneda, R. Kasai, O. Tanaka, K. Nishi, Chem. Pharm. Bull. 1977, 25, 2895].

The chloroform soluble fraction was submitted to column chromatography (silica gel, Hexane/CHCl₃ mixtures of increasing polarity) and twenty fractions (1-20) were collected. Compound 2 was obtained from Fr. 12 (EtOAc/hexane (45:55) and also from base hydrolysis of pulicarside 1 when it was refluxed with 5% aqueous KOH solution for 2 h. The mixture was then neutralized with a dilute HCl solution and extracted with n-butanol (3×6 ml). The combined n-butanol fractions were evaporated to gave aglycone having similar ¹H-NMR data as that of already reported ent-16,17-acetonyl-(−)-kauran-19-oic acid [M. S. Correa, G. M. S. P. Guilhon, L. M. Conserva, Fitoterapia 1998, LXIX, 277].

### Activity Testing

α-Glucosidase (E.C.3.2.1.20) enzyme inhibition assay was performed according to the slightly modified method of Matsui et al. α-glucosidase (E.C.3.2.1.20) from Saccharomyces species, purchased from Wako Pure Chemical Industries Ltd. (Wako 076-02841). The enzyme inhibition was measured spectrophotometrically at pH 6.9 and at 37° C. using 0.7 mM p-nitrophenyl-α-D-glucopyranoside (PNP-G) as a substrate and 500 m units/mL enzyme, in 50 mM sodium phosphate buffer containing 100 mM NaCl. 1-Deoxynojirimycin (0.425 mM) and acarbose (0.78 mM) were used as positive control. The increment in absorption at 400 nm, due to the hydrolysis of PNP-G by α-glucosidase, was monitored continuously on microplate spectrophotometer (Spectra Max Molecular Devices, USA).) [T. Matsui, C. Yoshimoto, K. Osajima, T. Oki, and Y. Osajima. Biosci. Biotech. Biochem., 1996, 60, 2019].

Table 1 Result of In vitro quantitative studies on compounds 1 and 2 against known α-glucosidase inhibitors.

TABLE 1
Result of In vitro quantitative studies on compounds
1 and 2 against known α-glucosidase inhibitors.

| Name of Substance | IC₅₀ ± SEM [μM] |
|---|---|
| Paniculoside IV (1) | 406.7 ± 20 |
| Eent-16,17-dihydroxy-(-)-kauran-19- | 62.2 ± 0.00 |
| oic acid (2) | |
| 1-Deoxynojirimycin (positive control | 425 ± 8.14 |
| for α-glucosidase) | |
| Acarbose (positive control for α- | 780 ± 0.028 |
| glucosidase) | |

A critical analysis of the chemical structure shows that when the ring is cleaved in case of the compound, 1 (paniculoside IV) showed inhibitory effect on the enzyme (IC₅₀ 406.7±20) and when the sugar molecule is replaced by the COO— group from the molecule in case of the compound, 2 (Ent-16, 17-dihydroxy-(−)-kauran-19-oic acid) (IC₅₀ 62.2±0.008) the compound also showed a promising inhibitory activity against the enzymes compared to pulicarside 1. So the COO— group is playing a crucial role for the inhibitory effect on the enzyme.

What is claimed is:

1. A method for the treatment of diabetes mellitus wherein a therapeutically effective amount of an α-glucosidase inhibitors, selected from a group comprising paniculoside IV and Ent-16, 17-dihydroxy-(−)-kauran-19-oic acid and their pharmaceutically acceptable derivates is chosen for use in humans and animals.

2. A pharmaceutical composition, which comprises the compound of claim 1 and a pharmaceutically acceptable vehicle for administration to humans and animals.
